(19) United States
(12) Patent Application Publication
(10) Pub. No.: US 2023/0145442 A1
(43) Pub. Date: May 11, 2023

(54) CONDENSATION PRODUCT FOR USE IN A METHOD FOR THE TREATMENT OF COVID-19

(71) Applicants: BASF SE, Ludwigshafen (DE); CHRISTIAN-ALBRECHTS-UNIVERSITAET ZU KIEL, Kiel (DE); DEUTSCHES PRIMATENZENTRUM GMBH, Göttingen (DE)

(72) Inventors: Guenter Scherr, Ludwigshafen (DE); Sandra Gloria Koenig, Ludwigshafen (DE); Ulrich Mrowietz, Kiel (DE); Stefan Poehlmann, Göttingen (DE)

(21) Appl. No.: 17/916,086

(22) PCT Filed: Mar. 31, 2021
(86) PCT No.: PCT/EP2021/058433

§ 371 Date: Sep. 30, 2022

(30) Foreign Application Priority Data
Apr. 1, 2020 (EP) 20167585.7

(51) Int. Cl. A61K 31/17; A61K 31/05; A61K 31/115; A61K 33/04

(52) U.S. Cl. CPC A61K 31/17; A61K 31/05; A61K 31/115; A61K 33/04

(57) ABSTRACT

The present invention relates to a condensation product obtainable by reaction of a1) at least one aromatic system or heteroaromatic system, a2) at least one carbonyl compound, a3) if appropriate at least one sulfonating agent, and a4) if appropriate at least one urea derivative, where the condensation product has a molecular weight Mw of at least 300 g/mol, for use in a method for the treatment of COVID-19. The invention further relates to a use of the condensation product in a therapeutic method for the treatment of COVID-19; to a use of the condensation product as disinfectant against the virus SARS-CoV-2; and to a use of the condensation product for the production of a medicament which is an antiviral agent against SARS-CoV-2.

The present invention relates to a condensation product obtainable by reaction of a1) at least one aromatic system or heteroaromatic system, a2) at least one carbonyl compound, a3) if appropriate at least one sulfonating agent, and a4) if appropriate at least one urea derivative, where the condensation product has a molecular weight Mw of at least 300 g/mol, for use in a method for the treatment of COVID-19.

The invention further relates to a use of the condensation product in a therapeutic method for the treatment of COVID-19; to a use of the condensation product as disinfectant against the virus SARS-CoV-2; and to a use of the condensation product for the production of a medicament which is an antiviral agent against SARS-CoV-2.

Object of the present invention was to find a substance for use in a method for the treatment of COVID-19.

The object was solved by a condensation product obtainable by reaction of a1) at least one aromatic system or heteroaromatic system, a2) at least one carbonyl compound, a3) if appropriate at least one sulfonating agent, and a4) if appropriate at least one urea derivative, where the condensation product has a molecular weight Mw of at least 300 g/mol, for use in a method for the treatment of COVID-19.

The object was also solved by a use of the condensation product in a therapeutic method for the treatment of COVID-19; by a use of the condensation product as disinfectant against the virus SARS-CoV-2; and by a use of the condensation product for the production of a medicament which is an antiviral agent against SARS-CoV-2.

COVID-19 (Coronavirus disease 2019) is usually means an infectious disease caused by severe acute respiratory syndrome coronavirus 2 (SARS-CoV-2). The disease was first identified in December 2019 in Wuhan, the capital of China's Hubei province, and has since spread globally, resulting in the 2019-20 coronavirus pandemic. Common symptoms include for example fever, cough, and shortness of breath. Other symptoms may include muscle pain, sputum production, diarrhea, sore throat, loss of smell, and abdominal pain. While the majority of cases result in mild symptoms, some progress to viral pneumonia and multi-organ failure.

The virus SARS-CoV-2 is usually spread through close contact and via respiratory droplets produced when people cough or sneeze. Respiratory droplets may be produced during breathing but the virus is not generally airborne. People may also contract COVID-19 by touching a contaminated surface and then their face. The virus can often survive on surfaces up to 72 hours. The standard method of diagnosis is by reverse transcription polymerase chain reaction (rRT-PCR) from a nasopharyngeal swab. The infection can also be diagnosed from a combination of symptoms, risk factors and a chest CT scan showing features of pneumonia.

SARS-CoV-2 is a member of the subgenus Sarbecovirus (beta-CoV lineage B). Its RNA sequence is approximately 30,000 bases in length. SARS-CoV-2 is unique among known betacoronaviruses in its incorporation of a polybasic cleavage site, a characteristic known to increase pathogenicity and transmissibility in other viruses. The spike protein of the virus may cause sufficient affinity to the angiotensin converting enzyme 2 (ACE2) receptors of human cells to use them as a mechanism of cell entry. SARS-CoV-2 has usually a higher affinity to human ACE2 than the original SARS virus strain.

The term treatment also comprises the prophylaxis, therapy or cure of the aforementioned disease.

The condensation products according to the invention can be administered to animals and humans, preferably mammals and humans, particularly preferably humans. The condensation products according to the invention can here be administered themselves as drugs, in mixtures with one another or mixtures with other drugs or in the form of pharmaceutical compositions. Consequently, the use of the condensation products according to the invention for the production of one or more medicaments for the prophylaxis and/or treatment of the aforementioned diseases or as an antiviral agent, pharmaceutical compositions comprising an efficacious amount of at least one condensation product according to the invention and the use of these pharmaceutical compositions for the prophylaxis and/or treatment of the aforementioned diseases are likewise a subject of the present invention.

The invention also relates to th use of the condensation for the production of a medicament which is an antiviral agent against SARS-CoV-2.

The medicament can be present in common administration forms, in particular in the form of a pill, tablet, lozenge, granules, capsule, hard or soft gelatin capsule, aqueous solution, alcoholic solution, oily solution, syrup, emulsion, suspension, suppository, pastille, solution for injection or infusion, ointment, tincture, cream, lotion, cosmetic powder, spray, of a transdermal therapeutic system, nasal spray, aerosol, aerosol mixture, microcapsule, implant, rod, patch or gel. Preferably, the pharmaceutical compositions can be present here in the form of an aqueous solution or a spray (e.g. nasal spray). Preferably the condensation product is administerd in form of a spray or an aqeuous solution.

The medicament may comprise an efficacious amount of at least one condensation product according to the invention and a physiologically tolerable vehicle.

Likewise, the medicament according to the invention can also be a constituent of healthcare products such as sunscreen creams, nasal sprays, mouthwashes, toothpastes, plasters, (wet) wipes, cleansing lotions or shampoos.

Depending on the administration form used, the condensation products according to the invention are processed with physiologically tolerable vehicles, which are known as such to the person skilled in the art, to give the medicaments. The vehicle must of course be tolerable in the sense that it is compatible with the other constituents of the composition and is not harmful to health for the patient (physiologically tolerable). The vehicle can be a solid or a liquid or both and is preferably formulated with the compound as an individual dose, for example as a tablet, which can comprise from 0.05 to 95% by weight of the active compound (condensation product according to the invention). Further pharmaceutically active substances can likewise be present. The pharmaceutical compositions according to the invention can be produced according to one of the known pharmaceutical methods, which essentially consist in mixing the constituents with pharmacologically tolerable vehicles and/or further excipients such as fillers, binders, lubricants, wetting agents, stabilizers et cetera.

Preferred pharmaceutical compositions in the context of the present invention are mentioned below.

In one embodiment of the present invention, ointments, creams, fatty creams, gels, lotions or powders according to the invention can in each case be comprised in the range from 0.1 to 5% by weight, preferably 0.2 to 3% by weight, of condensation products according to the invention, based on the respective ointment, cream, fatty cream, lotion or the respective gel or powder.

In one embodiment of the present invention, powders or concentrates according to the invention can be comprised in the range from 1 to 75% by weight, preferably 10 to 65% by weight of condensation product according to the invention, based on the respective powder or concentrate.

Creams according to the invention are customarily oil-in-water emulsions, ointments according to the invention are customarily water-in-oil emulsions. Ointments and creams according to the invention comprise, in addition to preferably purified water, one or more oil components and preferably one or more surface-active substances, for example one or more emulsifiers or protective colloids. Furthermore, ointments and fatty creams according to the invention—like other administration forms of the condensation products according to the invention as well—comprise preservatives such as, for example, sorbic acid.

Suitable oil components are natural and synthetic waxes, natural and synthetic oils such as, for example, nut oil, fish oil, olive oil and polymers such as, for example, polyacrylic acid, polydimethylsiloxane and polymethylphenylsiloxane.

Suitable surface-active substances are, for example, compounds of the general formula (IV)

CH₃—(CH₂)ₙ—X—R³ (IV)

where the variables are defined as follows:

n is an integer in the range from 0 to 20, is preferably an even number in the range from 2 to 16, and

X is divalent groups which carry at least one atom other than carbon and hydrogen, preferably nitrogen and particularly preferably oxygen, in particular —O— and —COO—,

R³ is selected from hydrogen,

C₁—C₁₀-alkyl groups such as, for example, methyl, ethyl, n-propyl, isopropyl, n-butyl, iso-butyl, sec-butyl, tert-butyl, n-pentyl, isopentyl, sec-pentyl, neopentyl, 1,2-di-methylpropyl, isoamyl, n-hexyl, isohexyl, sec-hexyl, n-heptyl, n-octyl, 2-ethylhexyl, n-nonyl, n-decyl; particularly preferably C₁—C₄-alkyl such as methyl, ethyl, n-propyl, iso-propyl, n-butyl, isobutyl, sec-butyl and tert-butyl,

—(CH₂—CH₂—O)ₘ—H, where m is an integer in the range from 1 to 100, preferably to 25,

CH₃—(CH₂)ₙ—X—(O—CH₂—CH₂)ₘ—, where X and n can in each case be different or preferably identical.

Furthermore, ointments and creams according to the invention—like other administration forms of the condensation products according to the invention as well—can comprise organic solvents such as, for example, propylene glycol and glycerol.

Preferred examples of surface-active substances are, for example, isopropyl tetradecanoate, cetyl alcohol, palmitic acid, stearic acid, polyoxyethylene 2-stearyl ether, α-n-dodecyl-ω-hydroxypolyoxyethylene having, on average, 10 ethylene oxide units, 2-phenoxyethanol, polyoxyethylene 21-stearyl ether.

Fatty creams according to the invention are customarily water-in-oil emulsions and comprise, in addition to preferably purified water, one or more oil components and preferably one or more surface-active substances, for example one or more emulsifiers or protective colloids.

Suitable oil components are, in addition to the oil components described above, natural and synthetic fats such as, for example, mono- or polyethylenically unsaturated fatty acid glycerides.

Furthermore, fatty creams according to the invention can comprise one or more of the following substances: methyl 4-hydroxybenzoate, propyl 4-hydroxybenzoate, aqueous sorbitol solution, tris[n-dodecylpoly(oxoethylene)-4]phosphate, cetylstearyl alcohol, hexyl laurate, vitamin F glycerol ester, dimethicone 350, calcium lactate pentahydrate.

Gels according to the invention can comprise, for example, polyacrylic acid, sodium hydroxide and butylhydroxyanisole, for example 4-methoxy-2-tert-butylphenol, 4-methoxy-3-tert-butylphenol and mixtures of the two aforementioned compounds.

Lotions according to the invention can comprise, for example, at least one of the substances mentioned below: glycerol, zinc oxide, talc, lecithin, highly disperse silica, isopropanol, methyl 4-hydroxybenzoate, carageenan, sodium salt and phosphoric acid esters of the general formula (V)

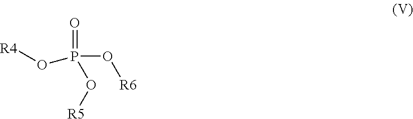

in which R⁴, R⁵ and R⁶ can be identical or different and are chosen from n—C₁₀—C₂₀-alkyl, in particular n-C₁₆—C₁₈-alkyl and H—(O—CH₂—CH₂)ₘ, where m is defined as above.

Cosmetic powders according to the invention can comprise, for example: calcium lactate pentahydrate, talc, cornstarch, 2-n-octyl-1-dodecanol, silica.

Powders according to the invention for the preparation of working solutions can comprise, for example, calcium lactate 5 H₂O and sodium sulfate (as a carrier material).

Concentrates according to the invention for the preparation of working solutions can comprise, for example: sodium salt of dodecylpoly(oxyethylene) 2-hydrogensulfate, sodium sulfate as a carrier material.

Instead of investigating ointments, creams, fatty creams, gels, lotions, cosmetic powders, powders or concentrates according to the invention for their efficacy, condensation products according to the invention, if appropriate as a stock solution, can be investigated for their efficacy. Suitable investigation methods are investigations on the inhibition of selected enzymes, for example human leucocyte elastase or protease plasmin. Furthermore, it can be investigated to what extent the replication of viruses in question is inhibited. Investigation methods of this type are described even more accurately in the following text (pharmacological investigations).

The invention further relates to the use of the condensation product as disinfectant against the virus SARS-CoV-2. In particular, the condensation products according to the invention are used as disinfectant in the hospital field, in particular hospital intensive care units, toilets, washrooms, households, food production or in stables or cages of animals, in particular of birds, pigs and cattle.

The disinfectants are thus not intended for administration as drugs, but they are suitable for the disinfection against the virus SARS-CoV-2 of, for example, the articles mentioned above. In the disinfectants according to the invention, at least one condensation product according to the invention is contained in the customary concentrations. Further components which are comprised in the disinfectants according to the invention are known to the person skilled in the art. Components of this type can vary, depending on the application area; the same applies for the concentration in the condensation product according to the invention.

The condensation product in usually present in dissolved form, preferably dissolved form in water, in the disinfectant.

The condensation product is usually obtainable by reaction of the following components:

Component a1), which is an at least one aromatic system or heteroaromatic system.

Aromatic systems are understood as meaning compounds having at least one phenyl ring, which can be substituted and which can also include a number of fused phenyl systems, for example naphthyl systems, phenanthrene systems and anthracene systems. If appropriate, in bi- or polycyclic systems individual cycles can also be completely or partly saturated, provided that at least one cycle is aromatic.

Heteroaromatic systems are described in the present invention as aromatic systems, which are preferably monocyclic or bicyclic, if appropriate also polycyclic. Examples of a heteroaromatic system are: pyrrole, furan, thiophene, imidazole, pyrazole, 1,2,3-triazole, 1,2,4-triazole, 1,3-oxazole (=oxazole), 1,2-oxazole (=isoxazole), oxadiazole, 1,3-thiazole (=thiazole), 1,2-thiazole (=isothiazole), tetrazole, pyridine, pyridazine, pyrimidine, pyrazine, 1,2,3-triazine, 1,2,4-triazine, 1,3,5-triazine, 1,2,4,5-tetrazine, indazole, indole, benzothiophene, benzofuran, benzothiazole, benzimidazole, quinoline, isoquinoline, quinazoline, cinnoline, quinoxaline, phthalazine, thienothiophene, 1,8-naphthyridine, other naphthyridines, purine or pteridine. Provided they are not monocyclic systems, in the case of each of the aforementioned heteroaromatic systems also the saturated form (perhydro form) or the partly unsaturated form (for example the dihydro form or tetrahydro form) or the maximally unsaturated (nonaromatic) form are additionally included for the second ring, provided the respective forms are known and stable. In the present invention, the description heteroaromatic system thus also comprises, for example, bi- or polycycles in which (in the case of the bicyclic system) both rings are aromatic, and bicyclic systems in which only one ring is aromatic. Such examples for heteroaromatic systems are: 3H-indoline, 2(1H)-quinolinone, 4-oxo-1,4-dihydroquinoline, 2H-1-oxoisoquinoline, 1,2-dihydroquinoline, 3,4-dihydroquinoline, 1,2-dihydroisoquinolinyl, 3,4-dihydroisoquinoline, oxindolyl, 1,2,3,4-tetrahydroisoquinoline, 1,2,3,4-tetrahydroquinoline, 5,6-dihydroquinoline, 5,6-dihydroisoquinoline, 5,6,7,8-tetra-hydroquinoline or 5,6,7,8-tetrahydroisoquinoline.

Preferably, at least one aromatic system or heteroaromatic system is selected from benzene, naphthalene, anthracene, aromatic alcohols, aromatic ethers and aromatic sulfones.

The aromatic or heteroaromatic system (component a1) can be unsubstituted or at least monosubstituted. If one or more substituents are present, these are independently of one another chosen from C₁—C₁₀-alkyl groups such as, for example, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁—C₄-alkyl such as methyl, ethyl, n-propyl, isopropyl, n-butyl, isobutyl, sec-butyl and tert-butyl,

C₂—C₁₀-alkenyl groups, in particular vinyl, 1-allyl, 3-allyl, 2-allyl, cis- or trans-2-butenyl, □-butenyl,

C₆—C₁₄-aryl groups aryl, such as, for example, phenyl, 1-naphthyl, 2-naphthyl, 1-anthryl, 2-anthryl, 9-anthryl, 1-phenanthryl, 2-phenanthryl, 3-phenanthryl, 4-phenanthryl and 9-phenanthryl, preferably phenyl, 1-naphthyl and 2-naphthyl, particularly preferably phenyl,

or benzyl groups.

Examples of preferred aromatic systems or heteroaromatic systems are:

benzene, toluene, ortho-xylene, meta-xylene, para-xylene, ethylbenzene, cumene, para-methylcumene, biphenyl, 2-methylbiphenyl, 3-methylbiphenyl, 4-methylbiphenyl, bitolyl (4,4′-dimethylbiphenyl), para-terphenyl, indene, fluorene, methylindenes (isomer mixture), naphthalene, 1-methylnaphthalene, 2-methylnaphthalene, 1,8-dimethylnaphthalene, 2,7-dimethylnaphthalene, phenanthrene, anthracene, 9-methylanthracene,9-phenylanthracene.

Examples of aromatic alcohols which may be mentioned are: phenol, ortho-cresol, meta-cresol, para-cresol, 2-ethylphenol, 3-ethylphenol, 4-ethylphenol, 2,3-dimethylphenol, 2,4-dimethylphenol, 2,5-dimethylphenol, 2,6-dimethylphenol, 3,4-dimethylphenol, 3,5-dimethylphenol, gallic acid, □-naphthol, □-naphthol, 9-hydroxyanthracene as a tautomer of anthrone, 9-hydroxyphenanthrene, diphenylmethane, phenyl-(2-methylphenyl)methane, phenylparatolylmethane, phenylmetatolylmethane.

Examples of aromatic ethers which may be mentioned are, for example: diphenyl ether, di-ortho-tolyl ether, di-meta-tolyl ether and di-para-tolyl ether.

Examples of aromatic sulfones which may be mentioned by way of example are diphenylsulfone and dihydroxydiphenylsulfone, in particular 4,4′-dihydroxydiphenylsulfone.

Component a1) is particularly preferably phenol.

In one embodiment of the present invention, mixtures of at least 2 aromatic systems are employed as component a1), for example mixtures of naphthalene and phenol, naphthalene and cresol (isomer mixture), naphthalene and diphenyl ether, naphthalene and ditolyl ether or phenol and ditolyl ether.

Component a2), which is at least one carbonyl compound. The carbonyl compound can be selected from aldehydes and ketones, preferably containing at least one aldehyde such as formaldehyde, acetaldehyde or propionaldehyde and in particular containing formaldehyde. If it is desired to employ formaldehyde, it is preferred to employ formaldehyde in aqueous solution.

Opitonal compounent a3), which is at least one sulfonating agent. Suitable sulfonating agents are, for example, sulfuric acid, in particular concentrated sulfuric acid, furthermore oleum having an SO₃ content of 1 to 30% by weight, furthermore chlorosulfonic acid and amidosulfonic acid. Concentrated sulfuric acid and oleum having an SO₃ content of 1 to 15% by weight are preferred.

Optional compound a4), which is at least one urea derivative. In principle, urea and all derivatives thereof are suitable as component a4). A urea derivative is preferred which carries at least one hydrogen atom on each nitrogen atom.

Particularly preferably, at least one urea derivative is chosen from compounds of the general formula (I)

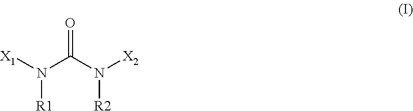

in which the variables are defined as follows:

X¹, X² are different or preferably identical and chosen from hydrogen and —CH₂OH, R¹, R² are different or preferably identical and are chosen from hydrogen, C₁—C₁₀-alkyl such as, for example, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; particularly preferably C₁-C₄-alkyl such as methyl, ethyl, n-propyl, isopropyl, n-butyl, isobutyl, sec-butyl and tert-butyl, or

R¹ and R² together form a C₂—C₁₀-alkylene unit, unsubstituted or substituted by 2 to 5 hydroxyl groups, such as, for example, —(CH₂)₂—, —CH₂—CH(CH₃)—, —(CH₂)₃—, —CH₂—CH(C₂H₅)—, —(CH₂)₄—, —(CH₂)₅—, —(CH₂)₆—, —(CH₂)₇—, —(CH₂)₈—, —(CH₂)₉—, —(CH—OH)₂-(cis or trans), preferably C₂—C₄-alkylene; in particular —(CH₂)₂—, —(CH₂)₃—, and —(CH—OH)₂-(cis or trans).

(Unsubstituted) urea, melamine or the cyclic urea derivatives of the formulae 1.1, 1.2 or 1.3 are very particularly preferred

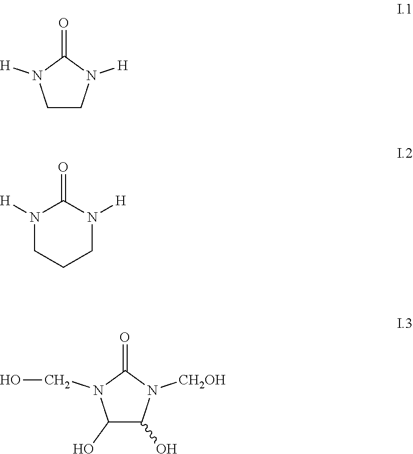 [SMILES: O=C1NCCN1 ; O=C1NCCCN1 ; O=C1N(CO)C(O)C(O)N1CO]

The condensation products may have an M_{w} value (weight-average molecular weight) of at least 300 g/mol, 500, 700, 1000, 1500, 2000, 3000, 4000, 5000, 7000, or 9000 g/mol.

The condensation products may have an M_{w} value (weight-average molecular weight) of up to 50 000 g/mol, 40 000, 30 000, 20 000, 15 000, 13 000, 11 000, 9000, 7000, 5000, 4000, 3000, or 2000 g/mol.

The condensation products may have an M_{w} value in the range from 300 to 50000 g/mol, preferably of 300 to 10 000 g/mol, and in particular of 1000 to 3000 g/mol.

M_{w} values in the context of the present invention are determined by GPC standard procedures, BASF DIN standard 55672-1; solvent THF. Such methods are illustrated in more detail in the examples. Preferably, the ratio M_{w}/Mₙ here is <10, in particular M_{w}/Mₙ <5 (M_{w}=weight-average molecular weight, Mₙ=number-average molecular weight).

The condensation products have usually a melting point above 100° C., perferably above 150° C.

The condensation products have are usually clear soluble in water at a concentration of 10 wt % at 23° C. in distilled water.

Processes for the preparation of the condensation product are known to the person skilled in the art, for example they are described in EP0037250, DE1113457, or DE848823.

The reaction can be carried out in one or in a number of steps. For example, it is possible first a1) to react at least one aromatic system or heteroaromatic system a3) if appropriate with at least one sulfonating agent and then to react it in the same vessel without prior isolation with a2) at least one carbonyl compound and a4) if appropriate at least one urea derivative.

In another embodiment, it is possible to proceed by a1) reacting at least one aromatic system or heteroaromatic system a3) with at least one sulfonating agent, isolating the product and then reacting it with the reaction product of a2) at least one carbonyl compound with a4) at least one urea derivative.

It is possible in one embodiment of the present invention to react reactants a1) and a2) and if appropriate a3) and a4) in one portion in each case.

In another embodiment of the present invention, at least one reactant a1) to a4) is reacted in at least two portions.

In a special embodiment of the present invention, a number of reactants a1) and a2) and if appropriate a3) and a4) are reacted in a number of portions.

In one embodiment of the present invention, during the reaction one or more further reactants a5) can be added, for example NaHSO₃, Na₂S₂O₅, KHSO₃, K₂S₂O₅, aqueous alkali metal hydroxide solution, in particular aqueous sodium hydroxide solution and aqueous potassium hydroxide solution, and aqueous ammonia. The further reactant a5) serves in particular for the adjustment of the pH and the control of the solubility of the final product.

In one embodiment of the present invention, a1) to a5) reactants are chosen in the following ratio:

a1) the aromatic system(s) in the range from altogether 10 to 70% by weight, preferably altogether 20 to 60% by weight, particularly preferably altogether 35 to 50% by weight,

a2) the aldehyde(s) or the ketone(s) in the range from altogether 5 to 40% by weight, preferably altogether 10 to 30% by weight, particularly preferably altogether 15 to 25% by weight,

a3) if appropriate the sulfonating agent(s) in the range from altogether 5 to 50% by weight, preferably altogether 10 to 40% by weight, particularly preferably altogether 20 to 30% by weight, sulfonating agents always being calculated as SO₃,

a4) the urea derivative(s) in the range from 0 to altogether 30% by weight, preferably altogether 10 to 25 and particularly preferably 15 to 25% by weight, where % by weight are in each case based on the sum of all reactants a1) and a2), if appropriate al) to a4),

a5) the additional reactant(s) in the range from 0 to altogether 30% by weight, preferably to altogether 25% by weight and particularly preferably altogether to 20% by weight, where the % by weight data from a5) are based on the sum of the reactants a1) and a2), if appropriate a1) to a4).

It is possible, for example, to react at temperatures in the range from 40 to 200° C., preferably 50 to 110° C. Customarily, the temperature of the reaction is adapted to a1) and a2). If it is desired, for example, to react aromatic alcohols, it is preferred to react at temperatures in the range from 50 to 110° C. Of course, it is also possible to set a certain temperature profile during the reaction. Thus it is possible, for example, first to start the reaction at 90 to 100° C. and after some time, for example, after 2 to 10 hours, to cool to 40 to 75° C. and to complete the reaction over a period of, for example, 1 to 10 hours.

Reaction is carried out, for example, at atmospheric pressure, but can, if desired, also be carried out at higher pressures, for example, 1.1 to 10 bar.

By means of the reaction described above, reaction solutions are obtained which customarily contain large amounts of acids such as, in particular, sulfuric acid or—in the case of the use of chlorosulfonic acid—HCl. Furthermore, reaction solutions can contain large amounts of alkali metal sulfate and/or alkali metal chloride.

Following the reaction described above, it is possible using, for example, aqueous alkali metal hydroxide solution or aqueous ammonia to set a pH in the range from 3 to 10, preferably 3.5 to 9.

By addition of water to reaction solutions obtainable by the reaction described above, it is possible by diluting with water to set a water content in the range from 70 to 95% by weight, preferably 75 to 90% by weight.

Consequent to the actual reaction and optionally consequent to the dilution with water, the reaction mixture obtainable by the reaction or the reaction solution obtainable by the reaction described above can be treated by molecular size-dependent separation processes. It is possible here to use one or more different molecular size-dependent separation processes or to carry out a molecular size-dependent separation process once or repeatedly. It is known to the person skilled in the art, how the preparation of the condensation product is to be controlled in order to obtain condensation products having high M_{w} values, such that the carrying out of molecular size-dependent separation processes is not obligatory.

### EXAMPLES

Solutions are understood as meaning aqueous solutions if not expressly specified otherwise. ppm relates to parts by weight.

The molecular weight determinations are carried out using gel permeation chromatography (GPC): Stationary phase: poly(2-hydroxymethacrylate) gel crosslinked with ethylene glycol dimethacrylate, obtainable commercially as HEMA BIO from PSS, Mainz, Germany. Eluent: mixture of 30% by weight of tetrahydrofuran (THF), 10% by weight of acrylonitrile, 60% by weight of 1 molar NaNO₃ solution

Internal standard: 0.001% by weight of benzophenone, based on eluent Flow: 1.5 ml/min Concentration: 1% by weight in the eluent containing internal standard Detection: UV/V is spectrometrically at 254 nm Calibration using polystyrene calibration part from PSS. Mₙ: number-average molecular weight in [g/mol] M_{w}: weight-average molecular weight in [g/mol]

For the determination of free formaldehyde, a flow injection apparatus according to Huber is employed, see Fresenius Z. Anal. Chem. 1981, 309, 389. The column chosen is a thermostatted reaction column 170×10 mm, filled with glass beads, which is operated at 75° C. The detector (continuous flow detector) is set at a wavelength of 412 nm. The procedure is as follows: For the preparation of a reagent solution, 62.5 g of ammonium acetate are dissolved in 500 ml of distilled water, 7.5 ml of concentrated acetic acid and 5.0 ml of acetylacetone are added and filled up to 1000 ml with distilled water. 0.1 g of the condensation product to be investigated is weighed into a 10 ml volumetric flask, filled up to 10 ml with distilled water and the respective sample solution is obtained. 100 μl of sample solution in each case are added, mixed with reagent solution and a mean residence time of 1.5 minutes is set, which corresponds to a flow of 35 ml/min.

For the determination of the absolute values, the flow injection apparatus is calibrated with formaldehyde solutions of known content.

### Example 1

Reactants were: a) phenol, b) concentrated sulfuric acid, c) formaldehyde, d) urea

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 0.34 kg of water of 20° C. and cooled to 70° C.

2.06 kg of aqueous urea solution (68% by weight) are metered in, the temperature rising to 95° C.; subsequently the mixture is cooled to 75° C.

4.10 kg of aqueous formaldehyde solution (30% by weight) are added over a period of 90 minutes, care being taken that the temperature does not rise above 75° C.

Subsequently, it is partially neutralized using 0.78 kg of aqueous sodium hydroxide solution (50% by weight), 0.30 kg of water are added, and the mixture is subsequently stirred for 30 minutes and cooled further.

1.36 kg of phenol are added at a temperature of 50° C. 1.14 kg of aqueous formaldehyde solution (30% by weight) are subsequently metered in at 50° C. over 20 minutes and the mixture is subsequently stirred for a further 30 minutes at 55° C.

The final adjustment of concentration and pH is carried out by addition of 1.40 kg of sodium hydroxide solution (50% by weight) and 2.5 kg of water. 18.5 kg of reaction solution 1.1 are obtained containing 43% by weight of nonvolatile fractions.

The analysis of the reaction solution affords the following values:

sodium sulfate by IC (based on nonvolatile fractions): 6.8% by weight; phenol by HPLC (based on nonvolatile fractions): 0.36% by weight; 4-phenolsulfonic acid by HPLC (based on nonvolatile fractions): 2.89% by weight; free formaldehyde: 75 ppm, based on nonvolatile fractions. Mₙ 890 g/mol, M_{w} 7820 g/mol, determined by GPC.

### Example 2

Reactants were: a) phenol, b) concentrated sulfuric acid, c) formaldehyde,

2.75 kg of phenol are introduced into a stirring apparatus and treated with 1.48 kg of concentrated sulfuric acid (96% by weight) for 20 minutes. Care is to be taken here that the temperature does not exceed 105° C. Subsequently, the reaction mixture is stirred at 100 to 105° C. for 3 hours and then cooled to 50° C.

2.00 kg of aqueous formaldehyde solution (30% by weight) are added over a period of approximately one hour, care being taken that the temperature does not exceed 55° C. Subsequently, the mixture is stirred at 50 to 55° C. for 10 hours, then 1.80 kg of water are added and it is finally stirred at 95 to 100° C. for 4 hours.

After cooling to room temperature, the final adjustment of concentration and pH is carried out by addition of aqueous sodium hydroxide solution (50% by weight) and water. 10.2 kg of reaction solution 1.2 are obtained containing 40% by weight of nonvolatile fractions.

The analysis of reaction solution affords the following values: sodium sulfate by IC (based on nonvolatile fractions): 15.4% by weight; phenol by HPLC (based on nonvolatile fractions): 0.11% by weight; 4-phenolsulfonic acid by HPLC (based on nonvolatile fractions): 5.34% by weight; free formaldehyde: 8 ppm, based on nonvolatile fractions. Mₙ 1810 g/mol, M_{w} 9040 g/mol, determined by GPC.

### Example 3

Reactants were a) phenol, b) concentrated sulfuric acid, c) formaldehyde, d) urea

2.04 kg of phenol are introduced into a stirring apparatus and treated with 2.48 kg of concentrated sulfuric acid (96% by weight) for 20 minutes. Care is to be taken here that the temperature does not exceed 105° C. Subsequently, the reaction mixture is stirred at 100 to 105° C. for 2 hours and then diluted with 340 g of water.

2.05 kg of urea solution (68% by weight) are metered in, care being taken that the temperature does not exceed 95° C.

3.60 kg of aqueous formaldehyde solution (30% by weight) are then added at 83 to 93° C. over a period of 1.5 hours.

After a stirring time of 15 minutes, 800 g of aqueous sodium hydroxide solution (50% by weight) are added, care being taken that the temperature does not exceed 85° C., so that the pH is subsequently between 7.3 and 7.5. 11.3 kg of reaction solution 1.3 containing 47% by weight of nonvolatile fractions are obtained.

The analysis of reaction solution affords the following values:

sodium sulfate by IC (based on nonvolatile fractions): 10.3% by weight; phenol by HPLC (based on nonvolatile fractions): 0.74% by weight; 4-phenolsulfonic acid by HPLC (based on nonvolatile fractions): 1.36% by weight; free formaldehyde: 99 ppm, based on nonvolatile fractions. Mₙ 1990 g/mol, M_{w} 17.020 g/mol, determined by GPC.

1. A condensation product obtained by reacting
b1) at least one compound selected from phenol and dihydroxydiphenylsulfone,
b2) at least one aldehyde selected from formaldehyde, acetaldehyde, and propionaldehyde,
b3) if appropriate concentrated sulfuric acid, and
b4) if appropriate at least one urea derivative selected from urea, melamine, 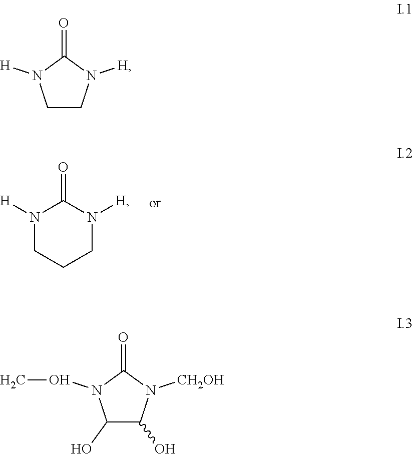
wherein the condensation product has a molecular weight Mw in a range of from 4,000 to 7,000 g/mol,
for use in a method for the treatment of COVID-19.

2. The condensation product according to claim 1, wherein the condensation product is obtained by reacting
a1) phenol,
a2) formaldehyde,
a3) concentrated sulfuric acid, and
a4) urea.

3. The method according to claim 5, where the condensation product is administered in form of a spray or an aqueous solution.

4. The method according to claim 5, where COVID-19 is an infectious disease caused by severe acute respiratory syndrome coronavirus 2 (SARS-CoV-2).

5. A therapeutic method for the treatment of COVID-19 comprising administration of a condensation product according to claim 1.

6. A method of disinfecting against the virus SARS-CoV-2 comprising applying the condensation product of claim 1.

7. The method according to claim 6 where the condensation product is present in dissolved form in water.

8. A method of producing a medicament, which is an antiviral agent against SARS-CoV-2, comprising adding a condensation product as defined in claim 1 to a physiologically tolerable vehicle.

9. (canceled)

10. (canceled)

11. (canceled)
